(19) United States
(12) Patent Application Publication
(10) Pub. No.: US 2011/0004249 A1
(43) Pub. Date: Jan. 6, 2011

(54) FLEXIBLE SPINAL FIXATION DEVICE AND ROD THEREOF

(75) Inventors: Ming-Chang Wu, Guishan Shiang (TW); Jia-Jyun Hong, Guishan Shiang (TW); Chi-Bin Wu, Guishan Shiang (TW)

Correspondence Address: Juan Carlos A. Marquez;c/o Stites & Harbison PLLC, 1199 North Fairfax Street, Suite 900, Alexandria, VA, 22314-1437, US

(73) Assignee: AccuMIS Inc., Zhonghe City (TW)

(21) Appl. No.: 12/534,164

(22) Filed: Aug. 3, 2009

(30) Foreign Application Priority Data
Jul. 3, 2009 (TW) 098122642

(51) Int. Cl. A61B 17/70

(52) U.S. Cl. 606/254; 606/246

(57) ABSTRACT

A flexible spinal fixation device and a rod thereof are provided. The flexible spinal fixation device includes at least two bone screws and a rod. The rod is fixedly coupled to the bone screws, and the bone screws are implanted into a patient's vertebrae. The rod has a flexible part and is thereby rendered flexible. After the flexible spinal fixation device is implanted into the patient's spine, the patient's spine can stoop as in a healthy condition. Moreover, vertebral stress around an injured vertebra and force between the bone screws and the rod can both be reduced. The flexible spinal fixation device also prevents premature vertebral aging and has an extended service life.

### BACKGROUND OF THE INVENTION

1. Technical Field

The present invention relates to a flexible spinal fixation device and a rod thereof. More particularly, the present invention relates to a flexible spinal fixation device and a rod thereof for use in spinal fixation.

2. Description of Related Art

The spine is composed of a plurality of bones, or vertebrae, connected together. A normal spine resembles a straight line when viewed from the front or back, and the spine can curve in three axial directions so as to enable various body movements. If a person's spine is displaced or deformed, the person's daily activities will be affected. In addition, as there are a multitude of nerves between vertebrae, a serious vertebral deformity will have direct impact on these spinal nerves and may lead to malfunction of organs at the distal ends of the spinal nerves.

In most cases, corrective surgery is indicated for patients with spinal deformity. During the corrective surgery, a plurality of bone screws are implanted into the deformed vertebrae from the side, and the bone screws are arranged in such a way as to present the normal position of the spine. Then, a rod is fixedly coupled to the bone screws to connect the bone screws together. By virtue of the rod, each two adjacent bone screws are kept at a fixed distance, thus supporting and correcting the deformed vertebrae.

To achieve the supportive and corrective effect, the rod must be made of a highly rigid material. As a result, the vertebrae fixed by the rod are not allowed to stoop freely, and the patient's mobility is restricted after the surgery. Moreover, as the number of rods implanted laterally into the vertebrae increases, the number of vertebrae that cannot stoop freely also increases, thus further limiting the range of movement of the spine as a whole.

In addition, when the number of vertebrae capable of normal movement decreases, these normal vertebrae that need not be fixed by the rod or rods will be subjected to significantly increased vertebral stress in order to maintain the original function of the spine. Consequently, the normal vertebrae may age prematurely or even be damaged due to overuse.

### BRIEF SUMMARY OF THE INVENTION

The present invention provides a flexible spinal fixation device and a rod thereof, wherein the rod has a flexible part that renders the rod flexible. Hence, a spine fixed by the spinal fixation device is still allowed to carry out normal activity, thereby improving a patient's quality of life after the surgical operation.

The present invention provides a flexible spinal fixation device and a rod thereof which allow normal activity of a spine, thereby reducing vertebral stress around an injured vertebra and preventing the remaining normal vertebrae from aging prematurely.

In order to achieve the above and other effects, the present invention provides a flexible spinal fixation device including: at least two bone screws for being fixed in vertebrae; and a rod formed with at least one flexible part and configured for being fixedly coupled to the bone screws.

In order to achieve the above and other effects, the present invention also provides a rod for spinal fixation, characterized in that the rod is formed with at least one flexible part.

Implementation of the present invention at least involves the following inventive steps:

1. With the spinal fixation device being flexible, a patient's spine can move as normal after the spinal surgery, thereby improving the patient's postoperative quality of life.

2. With the flexible rod, vertebral stress around an injured vertebra can be reduced, thereby preventing overuse of the remaining normal vertebrae.

### BRIEF DESCRIPTION OF THE SEVERAL VIEWS OF THE DRAWINGS

The invention as well as a preferred mode of use, further objectives, and advantages thereof will be best understood by referring to the following detailed description of illustrative embodiments in conjunction with the accompanying drawings, wherein:

FIG. 1 is a perspective view of a flexible spinal fixation device according to the present invention;

FIG. 2A is a perspective view of an embodiment of a rod according to the present invention;

FIG. 2B is a perspective view of another embodiment of the rod according to the present invention;

FIG. 3A is a perspective view of yet another embodiment of the rod according to the present invention;

FIG. 3B is a perspective view of still another embodiment of the rod according to the present invention; and

FIG. 4 is a perspective view showing application of the flexible spinal fixation device according to the present invention.

### DETAILED DESCRIPTION OF THE INVENTION

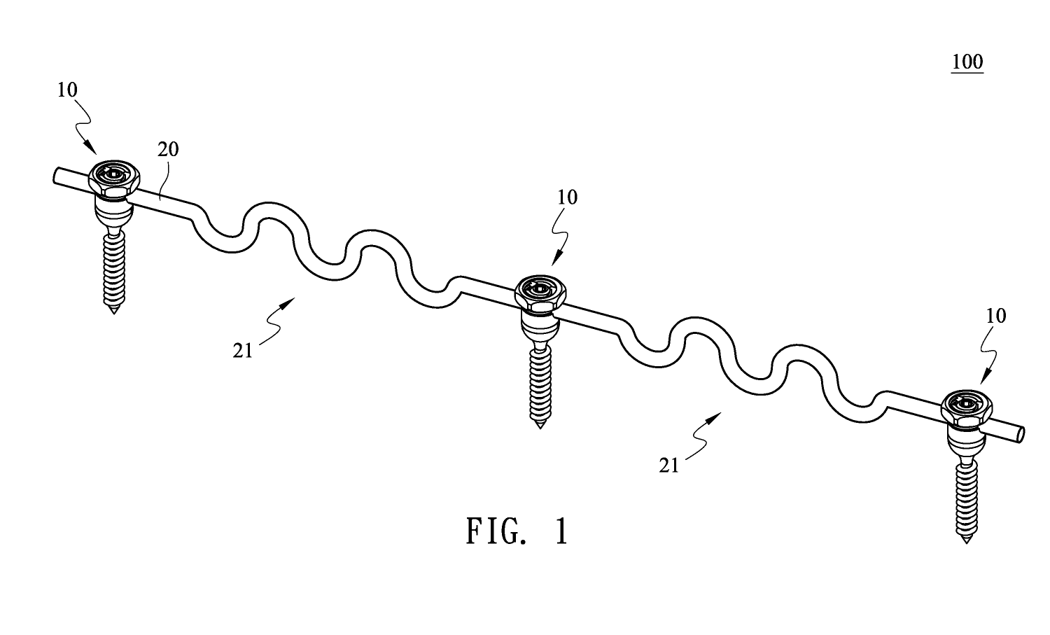

Referring to FIG. 1, a flexible spinal fixation device 100 according to an embodiment of the present invention includes at least two bone screws 10 and a rod 20.

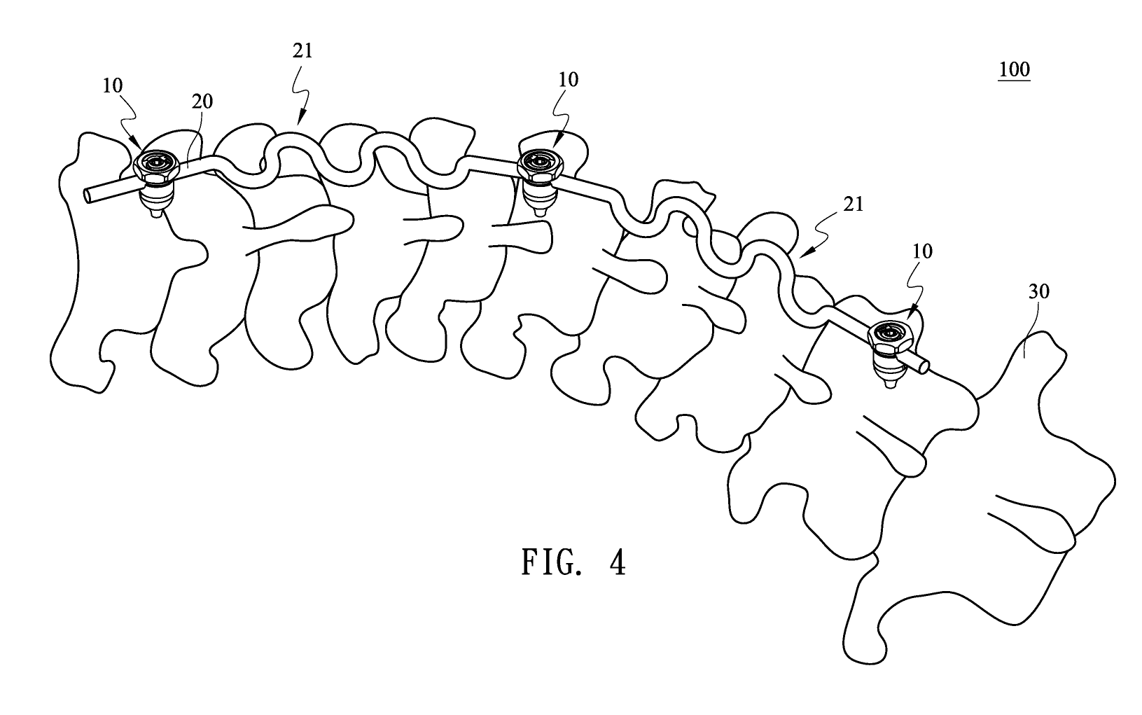

As shown in FIG. 1 and FIG. 4, the bone screws 10 are configured for being fixed in a spine 30, and the bone screws 10 are connected to one another by the rod 20. Therefore, when the bone screws 10 are used in conjunction with the rod 20, a pulling force is exerted on a lateral side of the spine 30 to fix the spine 30 at a normal angle, thereby correcting abnormal curvature of the spine 30. Each of the bone screws 10 is made of an alloy or a metal.

As shown in FIG. 1 and FIG. 4, the rod 20 is formed with at least one flexible part 21 which makes the rod 20 flexible. The rod 20 is fixedly coupled to the bone screws 10. Moreover, the rod 20 is made of an alloy or a metal so as to have high rigidity. When the rod 20 has two flexible parts 21, three bone screws 10 are used to support the rod 20, thereby increasing the range in which the spine 30 is fixed.

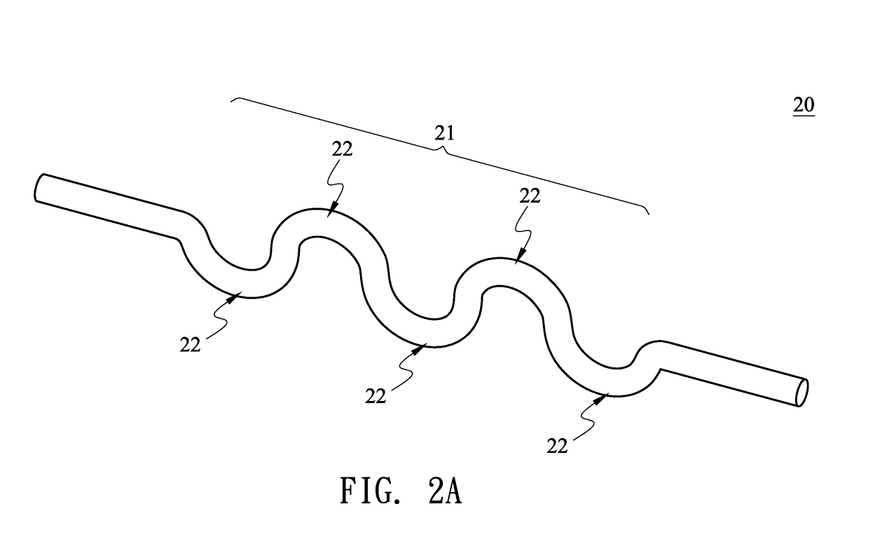

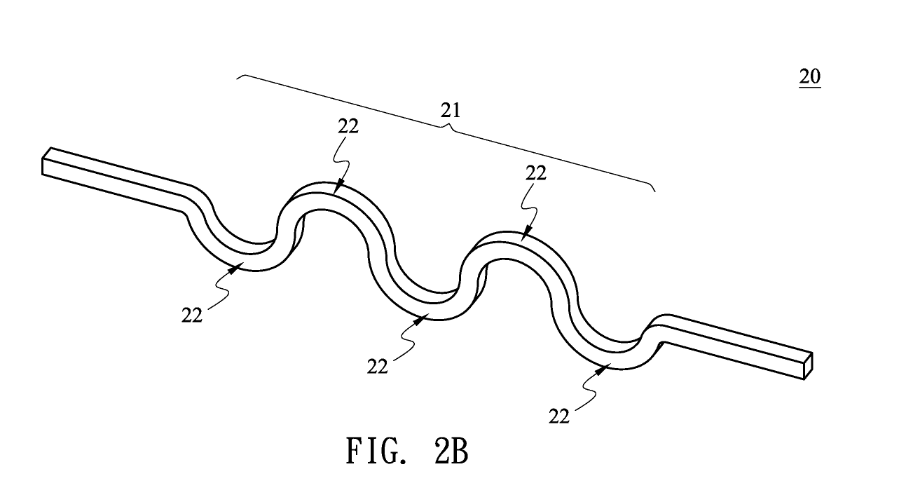

The rod 20 is in the form of a cylinder, as shown in FIG. 2A, or in the form of a rectangular prism, as shown in FIG. 2B. Each of the at least one flexible part 21 of the rod 20 is formed with at least one wavy unit 22. The at least one wavy unit 22 renders the rod 20 flexible.

Referring back to FIG. 4, when the rod 20 is used to fix a patient's spine 30, the flexibility of the rod 20 allows the spine 30 to move freely. Therefore, not only is the patient's postoperative quality of life improved, but also the normal vertebrae around the injured vertebra or vertebrae are protected from premature aging. Besides, the at least one flexible part 21 reduces the force between the bone screws 10 and the rod 20, thereby extending the service life of the spinal fixation device 100.

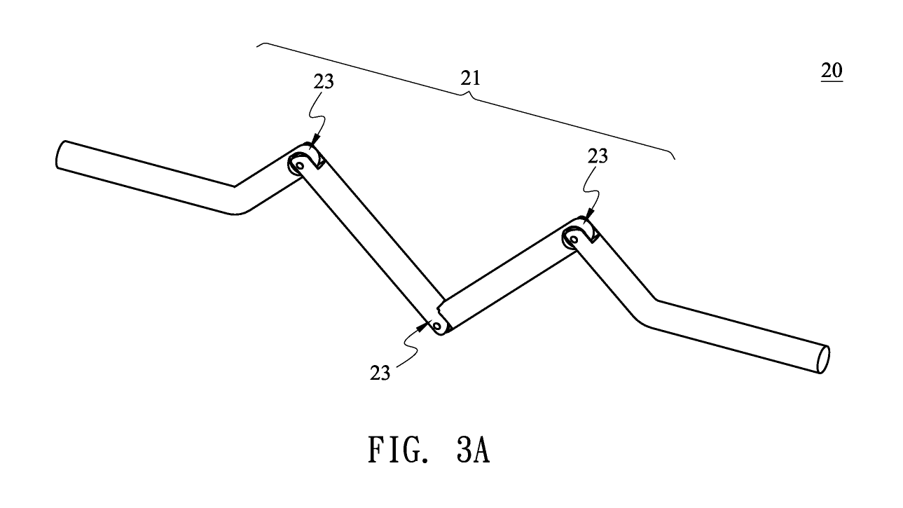

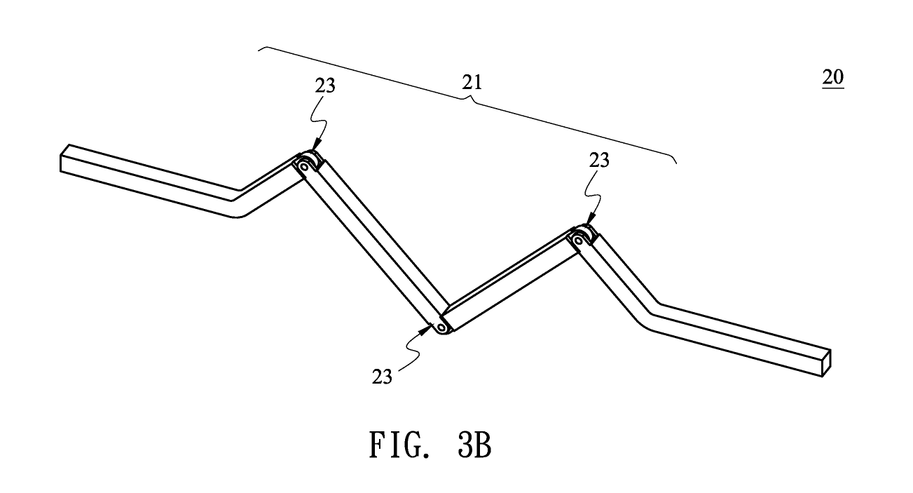

With reference to FIG. 3A and FIG. 3B, each of the at least one flexible part 21 of the rod 20 includes at least one pivot unit 23. The at least one pivot unit 23 also serves to make the rod 20 flexible. Therefore, according to practical needs, the at least one flexible part 21 of the rod 20 may include the wavy unit(s) 22, the pivot unit(s) 23, or a combination thereof.

The foregoing embodiments are illustrative of the characteristics of the present invention so as to enable a person skilled in the art to understand the disclosed subject matter and implement the present invention accordingly. The embodiments, however, are not intended to restrict the scope of the present invention. Hence, all equivalent modifications and variations made in the foregoing embodiments without departing from the spirit and principle of the present invention should fall within the scope of the appended claims.

What is claimed is:

1. A flexible spinal fixation device, comprising:
at least two bone screws for being fixed in vertebrae; and
a rod formed with at least a flexible part and configured for being fixedly coupled to the bone screws.

2. The spinal fixation device of claim 1, wherein each said bone screw is made of an alloy or a metal.

3. The spinal fixation device of claim 1, wherein the rod is made of an alloy or a metal.

4. The spinal fixation device of claim 1, wherein the rod is in form of a cylinder.

5. The spinal fixation device of claim 1, wherein the rod is in form of a rectangular prism.

6. The spinal fixation device of claim 1, wherein each said flexible part is formed with at least a wavy unit.

7. The spinal fixation device of claim 1, wherein each said flexible part is formed with at least a pivot unit.

8. A rod for spinal fixation, characterized in that the rod is formed with at least a flexible part.

9. The rod of claim 8, wherein the rod is made of an alloy or a metal.

10. The rod of claim 8, wherein the rod is in form of a cylinder.

11. The rod of claim 8, wherein the rod is in form of a rectangular prism.

12. The rod of claim 8, wherein each said flexible part is formed with at least a wavy unit.

13. The rod of claim 8, wherein each said flexible part is formed with at least a pivot unit.
